(19) United States
(12) Patent Application Publication
(10) Pub. No.: US 2026/0176419 A1
(43) Pub. Date: Jun. 25, 2026

(54) MANUFACTURING METHOD OF POLYESTER

(71) Applicant: NAN YA PLASTICS CORPORATION, Taipei City (TW)

(72) Inventors: Ching-Yao Yuan, Taipei City (TW); Cheng-Shui Huang, Taipei City (TW); Chung Yu Chen, Taipei City (TW); Mong-Chen Chang, Taipei City (TW); Zhang-Jian Huang, Taipei City (TW)

(73) Assignee: NAN YA PLASTICS CORPORATION, Taipei City (TW)

(21) Appl. No.: 19/067,983

(22) Filed: Mar. 2, 2025

(30) Foreign Application Priority Data
Dec. 23, 2024 (TW) 113150158

(51) Int. Cl. C08G 63/86; C08G 63/81; C08G 63/82; C08G 63/83; C08G 63/85; C08J 11/28

(52) U.S. Cl. CPC C08G 63/866; C08G 63/81; C08G 63/826; C08G 63/83; C08G 63/85; C08J 11/28

(57) ABSTRACT

A manufacturing method of a polyester includes: providing a recycled material, ethylene glycol, and a catalyst, in which the recycled material includes PET; performing a depolymerization step with use of the recycled material, the ethylene glycol, and the catalyst to obtain a BHET crude product; performing a purification step with use of the BHET crude product to obtain a BHET monomer; providing a phosphorus-containing monomer; and performing an esterification step and a polymerization step with use of the BHET monomer and the phosphorus-containing monomer.

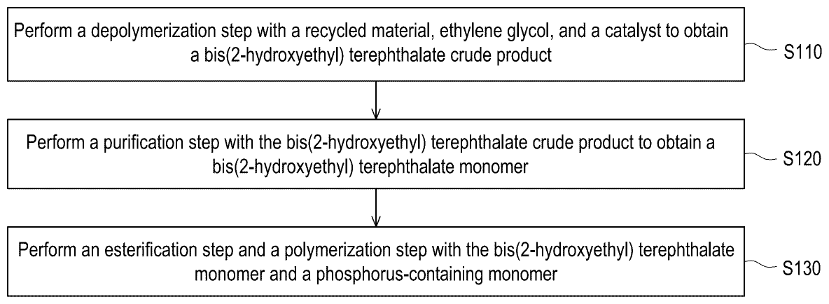

### CROSS-REFERENCE TO RELATED APPLICATION

This application claims the priority benefit of Taiwan application serial no. 113150158, filed on Dec. 23, 2024. The entirety of the above-mentioned patent application is hereby incorporated by reference herein and made a part of this specification.

### BACKGROUND

### Technical Field

The disclosure relates to a manufacturing method of a flame-resistant polyester.

### Related Art

Fabrics woven with materials such as flame-resistant polyesters are commonly used in products such as transmission cables for 3C (computer, communication, and consumer electronics). However, products manufactured using current polyester manufacturing methods face many problems, which reduce the product competitiveness. Therefore, an improved manufacturing method is desired for manufacturing polyesters.

### SUMMARY

The disclosure provides a manufacturing method of a polyester, which improves the product competitiveness.

A manufacturing method of a polyester according to an embodiment of the disclosure includes the following. A recycled material, ethylene glycol (EG), and a catalyst are provided, in which the recycled material includes polyethylene terephthalate. A depolymerization step is performed with use of the recycled material, the ethylene glycol, and the catalyst to obtain a bis(2-hydroxyethyl) terephthalate (BHET) crude product. A purification step is performed with use of the BHET crude product to obtain a BHET monomer. A phosphorus-containing monomer is provided. An esterification step and a polymerization step are performed with use of the BHET monomer and the phosphorus-containing monomer.

In an embodiment of the disclosure, the phosphorus-containing monomer is any one of

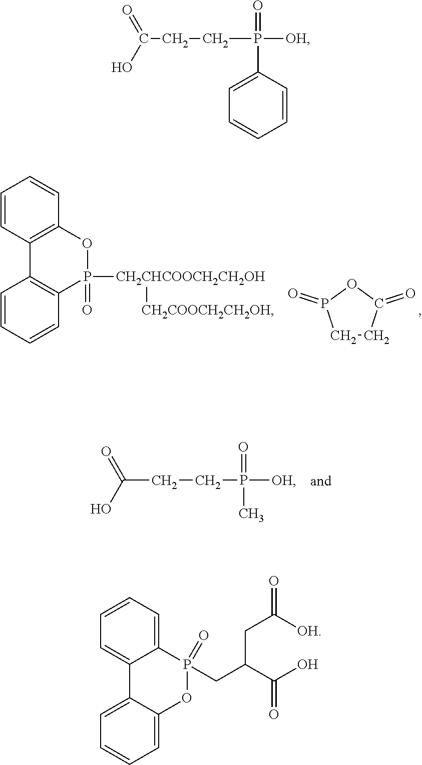

In an embodiment of the disclosure, an operating temperature of the depolymerization step is 180° C. to 220° C., an operating temperature of the purification step is 10° C. to 120° C., an operating temperature of the esterification step is 170° C. to 230° C., and an operating temperature of the polymerization step is 240° C. to 280° C.

In an embodiment of the disclosure, an operating time of the depolymerization step is 2 hours to 6 hours, an operating time of the purification step is 0.5 hours to 8 hours, an operating time of the esterification step is 0.2 hours to 3 hours, and an operating time of the polymerization step is 1 hour to 5 hours.

In an embodiment of the disclosure, in the depolymerization step, a weight ratio of the ethylene glycol to the recycled material used is 2 to 8.

In an embodiment of the disclosure, a molar ratio of the bis(2-hydroxyethyl) terephthalate monomer to the phosphorus-containing monomer used is 10 to 60.

In an embodiment of the disclosure, the catalyst includes an organic metal, an ionic liquid, or a combination thereof.

In an embodiment of the disclosure, the organic metal includes zinc acetate, cobalt acetate, tetrabutyl titanate, titanium citrate, antimony ethoxide, tri-n-octylaluminum, or a combination thereof, and the ionic liquid includes 1-butyl-3-methylimidazolium hexa-fluoro-phosphate (abbreviated as BMI-PF6), 1-butyl-3-methylimidazolium tetra-fluoro-borate (abbreviated as BMI-BF4), or a combination thereof.

In an embodiment of the disclosure, the purification step further includes an impurity adsorption procedure, and the impurity adsorption procedure uses activated carbon, polystyrene, activated clay, or a combination thereof as an adsorption material.

In an embodiment of the disclosure, the purification step further includes a crystallization procedure, a filtration procedure, and a drying procedure, and the crystallization procedure, the filtration procedure, and the drying procedure are performed sequentially after the impurity adsorption procedure.

Based on the above, the disclosure adopts the design of depolymerizing and purifying a recycled material into a BHET monomer, and then esterifying and polymerizing the BHET monomer with a phosphorus-containing monomer, which simultaneously solves the problems that occur in current physical mixing and chemical modification methods, such as poor processability, poor flame resistance, long thermal history and carbon footprint, poor hue, and lack of environmental friendliness. Thus, the disclosure produces a high-quality flame-resistant polyester with good product competitiveness.

To make the above-mentioned features of the disclosure more comprehensible, exemplary embodiments are provided below, along with detailed explanation in conjunction with the accompanying drawings.

### BRIEF DESCRIPTION OF THE DRAWINGS

The FIGURE is a partial flow chart showing a manufacturing method of a polyester according to an embodiment of the disclosure.

### DESCRIPTION OF THE EMBODIMENTS

In the following detailed description, exemplary embodiments disclosing specific details are set forth to provide a thorough understanding of various principles of the disclosure, for the purpose of illustration rather than limitation. It will be apparent to any person skilled in the art that, benefiting from the description, the disclosure may be practiced in other embodiments without these specific details disclosed herein. Besides, description of well-known devices, methods, and materials may be omitted to avoid obscuring the description of the various principles of the disclosure.

Reference is made to the drawings of this embodiment to more fully illustrate the disclosure. However, the disclosure may be embodied in various different forms and should not be construed as limited to the embodiments set forth herein.

Unless otherwise defined, all terms (including technical and scientific terms) used herein have the same meanings as commonly understood by any person skilled in the art to which the disclosure belongs.

The term “between” used in this specification for defining numerical ranges is intended to cover ranges equal to the stated endpoint values as well as ranges between the stated endpoint values. For example, a dimensional range between a first value and a second value may cover the first value, the second value, and any value between the first value and the second value.

The FIGURE is a partial flow chart showing a manufacturing method of a polyester according to an embodiment of the disclosure. Referring to the FIGURE, the manufacturing method of this embodiment includes at least the following steps.

First, a recycled material, ethylene glycol, and a catalyst are provided. The recycled material includes polyethylene terephthalate (PET). Then, as shown in step S110, a depolymerization step is performed with use of the recycled material, the ethylene glycol, and the catalyst to obtain a bis(2-hydroxyethyl) terephthalate (BHET) crude product. Here, the crude product includes a BHET dimer, a BHET trimer, a BHET oligomer, or the like.

Next, as shown in step S120, a purification step is performed with use of the BHET crude product to obtain a BHET monomer. In this step, the hue L of the BHET monomer may be greater than 90%, a may be between ±1, and b may be between ±2, but the disclosure is not limited thereto.

Then, a phosphorus-containing monomer is provided, and as shown in step S130, an esterification step and a polymerization step are performed with use of the BHET monomer and the phosphorus-containing monomer. The hue L of a flame-resistant polyester formed in this step may be greater than 80%, a may be between ±2, and b may be between ±4. Furthermore, the subsequent product may have an average filament breakage rate of less than 3 times/day, a spinning yield greater than 90%, and the flame resistance may be M1 level. Accordingly, in this embodiment, the design of depolymerizing and purifying the recycled material into the BHET monomer, and then esterifying and polymerizing the BHET monomer with the phosphorus-containing monomer simultaneously solves the problems that occur in current physical mixing and chemical modification methods, such as poor processability, poor flame resistance, long thermal history and carbon footprint, poor hue, and lack of environmental friendliness. Thus, the disclosure produces a high-quality flame-resistant polyester with good product competitiveness.

The flame-resistant polyester formed by current physical mixing methods is produced through co-extrusion, cooling, and pelletizing using an extruder. Consequently, the flame-resistant polyester is prone to filament breakage in subsequent spinning processes, resulting in poor processability. Additionally, due to poor mixing uniformity, the flame-resistant polyester also exhibits poor flame resistance. On the other hand, current chemical modification methods directly carry out esterification and copolymerization reactions with use of phthalic acid (PTA), ethylene glycol, and a phosphorus-containing monomer to form a flame-resistant polyester. This process often involves high temperatures over a long period of time, which leads to problems such as long thermal history and poor hue. Due to the long carbon footprint and the tendency to generate waste during the manufacturing process, it does not meet the environmental requirements. Since the manufacturing method of this embodiment does not utilize the physical mechanism of extrusion molding or utilize the chemical mechanism of phthalic acid as raw material, the copolymer structure formed by the manufacturing method of this embodiment solves the problems arising from these current mechanisms. Nevertheless, the disclosure is not limited thereto.

The following will sequentially illustrate the possible specific details for implementing the above-mentioned steps. However, the description is not intended to limit the disclosure.

### <Depolymerization Step>

In some embodiments, the effects may be improved under favorable operating conditions. For example, the operating temperature of the depolymerization step may be 180° C. to 220° C. and/or the operating time of the depolymerization step may be 2 hours to 6 hours. More preferably, the operating conditions may include an operating temperature of 190° C. to 210° C. and/or an operating time of 2 hours to 5 hours. However, the disclosure is not limited thereto.

In some embodiments, in the depolymerization step, the weight ratio of ethylene glycol to recycled material used is 2 to 8, and preferably 3 to 6. However, the disclosure is not limited thereto. Here, the weight ratio of ethylene glycol to recycled material used is the weight of ethylene glycol used divided by the weight of recycled material used, which means that the weight of ethylene glycol used is greater than the weight of recycled material used.

In some embodiments, the source of the recycled material may be recycled films, recycled PET bottles, recycled fibers, or the like.

In some embodiments, the catalyst includes an organic metal, an ionic liquid, or a combination thereof. For example, the organic metal may include zinc acetate, cobalt acetate, tetrabutyl titanate, titanium citrate, antimony ethoxide, tri-n-octylaluminum, or a combination thereof. The ionic liquid may include 1-butyl-3-methylimidazolium hexa-fluoro-phosphate, 1-butyl-3-methylimidazolium tetra-fluoro-borate, or a combination thereof. However, the disclosure is not limited thereto.

### <Purification Step>

In some embodiments, the effects may be improved under favorable operating conditions. For example, the operating temperature of the purification step may be 10° C. to 120° C. and/or the operating time of the purification step may be 0.5 hours to 8 hours. However, the disclosure is not limited thereto.

In some embodiments, the purification step includes an impurity adsorption procedure, and the impurity adsorption procedure uses activated carbon, polystyrene, activated clay, or a combination thereof as the adsorption material.

In some embodiments, the purification step further includes a crystallization procedure, a filtration procedure, and a drying procedure, and the crystallization procedure, the filtration procedure, and the drying procedure are performed sequentially after the impurity adsorption procedure. For example, the crystallization procedure may lower the temperature to 10° C. to 45° C., and preferably 20° C. to 35° C. However, the disclosure is not limited thereto.

### <Esterification Step>

In some embodiments, the effects may be improved under favorable operating conditions. For example, the operating temperature of the esterification step may be 170° C. to 230° C. and/or the operating time of the esterification step may be 0.2 hours to 3 hours. More preferably, the operating conditions may be set so that the operating temperature of the esterification step is 180° C. to 220° C. and/or the operating time of the esterification step is 0.5 hours to 2 hours. However, the disclosure is not limited thereto.

In some embodiments, the esterification pressure may also be controlled to be 1 bar to 3 bar. However, the disclosure is not limited thereto.

In some embodiments, the molar ratio of BHET monomer to phosphorus-containing monomer used is 10 to 60, for example, 10 to 30, or for example, 15 to 20. However, the disclosure is not limited thereto. Here, the molar ratio of BHET monomer to phosphorus-containing monomer used is the number of moles of the BHET monomer used divided by the number of moles of the phosphorus-containing monomer used, which means that the number of moles of the BHET monomer used is greater than the number of moles of the phosphorus-containing monomer used.

In some embodiments, the phosphorus-containing monomer is any one of

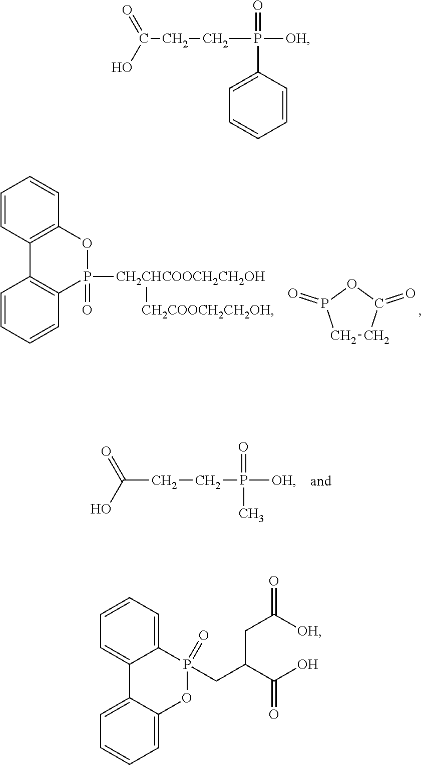

to achieve better reactivity. However, the disclosure is not limited thereto.

In some embodiments, the esterification step may optionally further use a catalyst. The catalyst is, for example, antimony ethylene glycol or the like. However, the disclosure is not limited thereto.

### <Polymerization Step>

In some embodiments, the effects may be improved under favorable operating conditions. For example, the operating temperature of the polymerization step may be 240° C. to 280° C. and/or the operating time of the polymerization step may be 1 hour to 5 hours. More preferably, the operating conditions may be set so that the operating temperature of the polymerization step is 250° C. to 270° C. and/or the operating time of the polymerization step is 2 hours to 4 hours. However, the disclosure is not limited thereto.

In some embodiments, the polymerization pressure may also be controlled to be 0.2 torr to 2 torr, and more preferably 0.5 torr to 1.5 torr. However, the disclosure is not limited thereto.

Examples and comparative examples are provided hereinafter to show the effects of the disclosure. Nevertheless, the claims of the disclosure are not limited to the scope of the examples.

The flame-resistant polyesters produced in the examples and comparative examples were evaluated according to the following method.

Intrinsic viscosity (IV): The specific viscosity of the polymer solution as the concentration approached zero.

Phosphorus content: Analyzed and quantified using LC (liquid chromatography) instrument to calculate the total molecular weight of phosphorus/molecular weight of the flame-resistant polyester in the flame-resistant polyester structure.

Hue: The CIE Lab color space defined by the International Commission on Illumination was adopted. The Lab color space is a color-opponent space with the L dimension representing lightness (also called the whiteness of the color), and a and b representing the color-opponent dimensions, based on non-linearly compressed CIE XYZ color space coordinates.

Yield: The weight of qualified products after spinning/weight of polyester.

Flame resistance: According to NF P 92-507 standard.

The polyesters in Examples 1 to 6 were manufactured by the following method.

Corresponding to step S110, a depolymerization step was performed with use of 1000 kg of PET bottle flakes (recycled polyethylene terephthalate material), 4000 kg of ethylene glycol, and 8 kg of zinc acetate (catalyst) to obtain a BHET crude product. Specifically, the depolymerization step was performed in a reaction vessel with PET bottle flakes, ethylene glycol, and zinc acetate under operating conditions of a temperature of 195° C. and a duration of 4 hours.

Corresponding to step S120, a purification step was performed with use of the obtained BHET crude product to obtain a BHET monomer. Specifically, the purification step used an impurity adsorption procedure, a crystallization procedure, a filtration procedure, and a drying procedure. First, the impurity adsorption procedure was performed, in which the temperature was lowered from 195° C. to 120° C., then 80 kg of activated carbon (adsorption material) was added and stirred for 30 minutes, followed by distilling out 2000 kg of ethylene glycol, and then activated carbon and impurities were filtered out using a 0.1 μm filter bag. After performing the impurity adsorption procedure, the crystallization procedure was performed, in which the filtrate was first cooled from 120° C. to 80° C., then 4000 kg of water was added and stirred for 30 minutes, and then cooled from 80° C. to 10° C., causing the BHET to crystallize out. Next, the filtration procedure and the drying procedure were performed, using a 5 μm filter to obtain a wet BHET solid filter cake, which was then dried at 80° C. and 200 torr for 2 hours to obtain 1200 kg of BHET monomer (hue: L was 94.5%, a was 0.4, and b was 1.7).

Corresponding to step S130, an esterification step and a polymerization step were performed with use of 1200 kg of the obtained BHET monomer and 45 kg of the phosphorus-containing monomer (compounds 1 to 5) from Table 1 to obtain 890 kg of flame-resistant polyester for Examples 1 to 6. Specifically, under operating conditions of a temperature of 195° C., a pressure of 760 torr, and a duration of 1 hour, the esterification step was performed by placing the BHET monomer, phosphorus-containing monomer, and 9 kg of catalyst (antimony ethylene glycol) in a three-neck glass flask. After performing the esterification step, the polymerization step was performed under operating conditions of a temperature of 260° C., a pressure of 0.6 torr, and a duration of 2.5 hours. In Table 1, compound 1 is

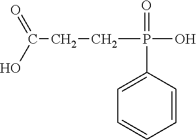 [SMILES: O=C(O)CCP(=O)(O)c1ccccc1]

commercially named Phosgard PF100®, compound 2 is

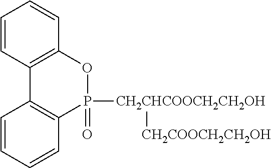

commercially named Ukanol ES®, compound 3 is commercially named

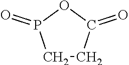

Exolit PE100®, compound 4 is

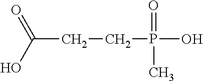

commercially named ring-opened Exolit PE100®, and compound 5 is

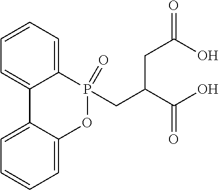 [SMILES: O=C(O)CC(CP1(=O)Oc2ccccc2-c2ccccc21)C(=O)O]

commercially named DOPO-Itaconic acid.

TABLE 1

| | | Example 1 | Example 2 | Example 3 | Example 4 | Example 5 | Example 6 |
|---|---|---|---|---|---|---|---|
| Flame-resistant | Type | Compound | Compound | Compound | Compound | Compound | Compound |
| monomer | | 1 | 1 | 2 | 3 | 4 | 5 |
| | Weight | 46.4 | 62.2 | 94.1 | 26.8 | 36.9 | 95 |
| | (kg) | | | | | | |

The characteristics and spinning evaluation of the flame-resistant polyesters of Examples 1 to 6 are shown in Table 2.

TABLE 2

| | | Example | Example | Example | Example | Example | Example |
|---|---|---|---|---|---|---|---|
| | | 1 | 2 | 3 | 4 | 5 | 6 |
| Flame-resistant | Intrinsic viscosity | 0.56 | 0.55 | 0.53 | 0.51 | 0.51 | 0.52 |
| polyester | (dl/g) | | | | | | |
| characteristics | Phosphorus content | 6680 | 8940 | 6940 | 6770 | 7010 | 8040 |
| | (ppm) | | | | | | |
| | L (%) | 85.6 | 83.4 | 82.1 | 81.9 | 81.3 | 80.4 |
| | a | 0.8 | 1.1 | 1.3 | 0.9 | 1.0 | 0.8 |
| | b | 2.2 | 2.8 | 3.1 | 3 | 3.2 | 3.7 |
| Spinning | Yield (%) | 98.2 | 96.4 | 97.3 | 98.2 | 95.9 | 98.1 |
| evaluation | Flame resistance | M1 | M1 | M1 | M1 | M1 | M1 |

The polyesters of Comparative Examples 1 to 6 were manufactured by the following method.

### Comparative Example 1

890 kg of virgin polyethylene terephthalate resin (IV of 0.83 dl/g, L of 83.4%, a of 0.3, b of 0.8) and 4.6 kg of flame retardant DOPO (9,10-dihydro-9-oxa-10-phosphaphenanthrene 10-oxide, CAS No: 35948-25-5) were fed into a twin-screw extruder and extruded at a temperature of 260° C., and then the molten resin was filtered through a 60 μm mesh. Subsequently, cooling and pelletizing procedures were performed to obtain the polyester of Comparative Example 1.

### Comparative Example 2

Comparative Example 2 differed from Comparative Example 1 only in that the flame retardant of Comparative Example 1 was changed to DPPE (1,2-Bis(diphenylphosphino) ethane, CAS No: 1663-45-2), and the weight used was 57.3 kg, to obtain the polyester of Comparative Example 2.

### Comparative Example 3

865 kg of terephthalic acid (PTA), 338 kg of ethylene glycol, 51.3 kg of flame retardant (compound 1, Phosgard PF100®) and 9 kg of catalyst (antimony ethylene glycol) were placed in a reaction vessel. The esterification reaction took place under operating conditions of a temperature of 260° C., a pressure of 2.8 torr, and a duration of 1.5 hours, while simultaneously distilling out the water generated from esterification, to obtain an oligomer of BHET. Subsequently, the polymerization reaction took place under operating conditions of a temperature of 260° C., a pressure of 0.6 torr, and a duration of 2.5 hours, while simultaneously distilling out the ethylene glycol generated from polymerization, to obtain 934 kg of the polyester of Comparative Example 3.

### Comparative Example 4

Comparative Example 4 differed from Comparative Example 3 only in that the flame retardant of Comparative Example 2 was changed to compound 2 (Ukanol ES®), and the weight used was 96.4 kg, to obtain 928 kg of the polyester of Comparative Example 4.

### Comparative Example 5

Comparative Example 5 differed from Comparative Example 1 only in that the virgin polyethylene terephthalate resin of Comparative Example 1 was changed to recycled PET bottles (IV of 0.81 dl/g, L of 71.3%, a of 0.2, b of 0.6), and the weight of flame retardant used was 48.4 kg, to obtain the polyester of Comparative Example 5.

### Comparative Example 6

Comparative Example 6 differed from Comparative Example 5 only in that the flame retardant of Comparative Example 5 was changed to DPPE (CAS No: 1663-45-2), and the weight used was 51.7 kg, and the recycled PET bottles had an IV of 0.81 dl/g, L of 70.1%, a of 0.3, and b of 0.8, to obtain the polyester of Comparative Example 6.

The characteristics and spinning evaluation of the polyesters of Comparative Examples 1 to 6 are shown in Table 3.

TABLE 3

| | | Comparative | Comparative | Comparative | Comparative | Comparative | Comparative |
|---|---|---|---|---|---|---|---|
| | | Example 1 | Example 2 | Example 3 | Example 4 | Example 5 | Example 6 |
| Characteristics | Intrinsic | 0.54 | 0.53 | 0.55 | 0.56 | 0.52 | 0.51 |
| of polyester | viscosity | | | | | | |
| | (dl/g) | | | | | | |
| | Phosphorus | 6820 | 9020 | 6890 | 6740 | 7210 | 8120 |
| | content (ppm) | | | | | | |
| | L (%) | 56.3 | 57.2 | 54.5 | 53.4 | 49.1 | 50.1 |
| | a | 2.3 | 2.8 | 1.4 | 1.7 | 3.2 | 3.4 |
| | b | 6.5 | 8.5 | 7.8 | 9.2 | 6.8 | 7.4 |
| Spinning | Yield (%) | 92.3 | 93.4 | 95.8 | 96.4 | 94.2 | 93.5 |
| evaluation | Flame | M2 | M2 | M1 | M1 | M2 | M2 |
| | resistance | | | | | | |

From the results in Table 2 and Table 3, the following conclusions can be drawn: Examples 1 to 6 had good spinning processability, excellent flame resistance, low carbon, and good hue. In contrast, Comparative Examples 1 to 2 and Comparative Examples 5 to 6 used physical mixing mechanisms, which did not provide uniformity as good as chemical bonding; and Comparative Examples 3 to 4 used chemical modification mechanisms, which underwent high thermal history temperatures. Especially when the reaction lasted for more than 2.5 hours with the esterification temperature and polymerization temperature both at 260° C., the flame-resistant polyesters in Comparative Examples 1 to 6 all had problems with quality, spinning processability, and poor hue.

To sum up, the disclosure adopts the design of depolymerizing and purifying a recycled material into a BHET monomer, and then esterifying and polymerizing the BHET monomer with a phosphorus-containing monomer, which simultaneously solves the problems that occur in current physical mixing and chemical modification methods, such as poor processability, poor flame resistance, long thermal history and carbon footprint, poor hue, and lack of environmental friendliness. Thus, the disclosure produces a high-quality flame-resistant polyester with good product competitiveness.

Although the disclosure has been described with reference to the embodiments above, they are not intended to limit the disclosure. Any person skilled in the art may make modifications and changes without departing from the spirit and scope of the disclosure. Therefore, the protection scope of the disclosure should be defined by the appended claims.

What is claimed is:

1. A manufacturing method of a polyester, comprising:
providing a recycled material, ethylene glycol, and a catalyst, wherein the recycled material comprises polyethylene terephthalate;
performing a depolymerization step with use of the recycled material, the ethylene glycol, and the catalyst to obtain a bis(2-hydroxyethyl) terephthalate crude product;
performing a purification step with use of the bis(2-hydroxyethyl) terephthalate crude product to obtain a bis(2-hydroxyethyl) terephthalate monomer;
providing a phosphorus-containing monomer; and
performing an esterification step and a polymerization step with use of the bis(2-hydroxyethyl) terephthalate monomer and the phosphorus-containing monomer.

2. The manufacturing method of the polyester according to claim 1, wherein the phosphorus-containing monomer any one of

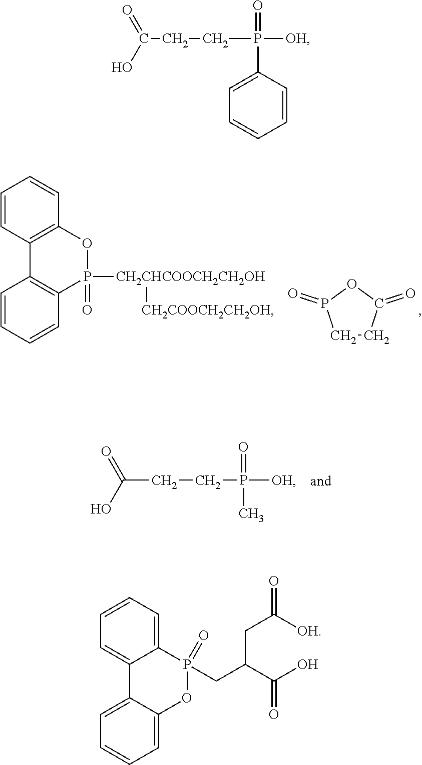

3. The manufacturing method of the polyester according to claim 1, wherein an operating temperature of the depolymerization step is 180° C. to 220° C., an operating temperature of the purification step is 10° C. to 120° C., an operating temperature of the esterification step is 170° C. to 230° C., and an operating temperature of the polymerization step is 240° C. to 280° C.

4. The manufacturing method of the polyester according to claim 1, wherein an operating time of the depolymerization step is 2 hours to 6 hours, an operating time of the purification step is 0.5 hours to 8 hours, an operating time of the esterification step is 0.2 hours to 3 hours, and an operating time of the polymerization step is 1 hour to 5 hours.

5. The manufacturing method of the polyester according to claim 1, wherein in the depolymerization step, a weight ratio of the ethylene glycol to the recycled material used is 2 to 8.

6. The manufacturing method of the polyester according to claim 1, wherein a molar ratio of the bis(2-hydroxyethyl) terephthalate monomer to the phosphorus-containing monomer used is 10 to 60.

7. The manufacturing method of the polyester according to claim 1, wherein the catalyst comprises an organic metal, an ionic liquid, or a combination thereof.

8. The manufacturing method of the polyester according to claim 7, wherein the organic metal comprises zinc acetate, cobalt acetate, tetrabutyl titanate, titanium citrate, antimony ethoxide, tri-n-octylaluminum, or a combination thereof, and the ionic liquid comprises 1-butyl-3-methylimidazolium hexa-fluoro-phosphate, 1-butyl-3-methylimidazolium tetra-fluoro-borate, or a combination thereof.

9. The manufacturing method of the polyester according to claim 1, wherein the purification step further comprises an impurity adsorption procedure, and the impurity adsorption procedure uses activated carbon, polystyrene, activated clay, or a combination thereof as an adsorption material.

10. The manufacturing method of the polyester according to claim 9, wherein the purification step further comprises a crystallization procedure, a filtration procedure, and a drying procedure, and the crystallization procedure, the filtration procedure, and the drying procedure are performed sequentially after the impurity adsorption procedure.
